(19) United States
(12) Patent Application Publication
(10) Pub. No.: US 2007/0255139 A1
(43) Pub. Date: Nov. 1, 2007

(54) User interface for automatic multi-plane imaging ultrasound system

(75) Inventors: Harald Deschinger, Frankenmarkt (AT); Peter Falkensammer, Voecklabruck (AT); Franz Gabeder, Aurach am Honga (AT)

Correspondence Address: Dean D. Small;The Small Patent Law Group LLP, Ste. 1611, 611 Olive Street, St. Louis, MO, 63101, US

(73) Assignee: GENERAL ELECTRIC COMPANY

(21) Appl. No.: 11/434,445

(22) Filed: May 15, 2006

Related U.S. Application Data
(60) Provisional application No. 60/795,535, filed on Apr. 27, 2006.

(51) Int. Cl. A61B 8/00; G06K 9/00

(52) U.S. Cl. 600/443; 382/128

(57) ABSTRACT

A diagnostic ultrasound system is provided for automatically displaying multiple planes from a 3-D ultrasound data set. The system comprises a user interface for designating a reference plane, wherein the user interface provides a safe view position option and a restore reference plane option. A processor module maps the reference plane into a 3D ultrasound data set and automatically calculates image planes based on the reference plane for a current view position and a prior view position. A display is provided to selectively display the image planes associated with the current and prior reference planes. Memory stores the prior reference plane in response to selection of the save reference plane option, while the display switches from display of the current reference plane to restore the prior reference plane in response to selection of the restore reference plane option. Optionally, the memory may store coordinates in connection with the current and prior reference planes.

### RELATED APPLICATION

The present application relates to and claims priority from Provisional Application Ser. No. 60/795,535 filed Apr. 27, 2006 titled “USER INTERFACE FOR AUTOMATIC MULTI-PLANE IMAGING ULTRASOUND SYSTEM”, the complete subject matter of which is hereby expressly incorporated in its entirety.

### BACKGROUND OF THE INVENTION

Embodiments of the present invention relate generally to systems and methods for automatically displaying multiple planes from 3-D ultrasound data sets, and more specifically for providing a user interface that affords an easy exchange and restoration of prior view positions.

Ultrasound systems are used in a variety of applications and by individuals with varied levels of skill. In many examinations, operators of the ultrasound system review select combinations of ultrasound images in accordance with predetermined protocols. In order to obtain the desired combination of ultrasound images, the operator steps through a sequence of operations to identify and capture one or more desired image planes. At least one ultrasound examination process has been proposed, generally referred to in as automated multi-planar imaging that seeks to standardize acquisition and display of the predetermined image planes. In accordance with this recently proposed ultrasound process, a volumetric image is acquired in a standardized manner and a reference plane is identified. Based upon the reference plane, multiple image planes are automatically obtained from the acquired volume of ultrasound information without detailed intervention by the user to identify individually the multiple image planes.

However, conventional ultrasound systems have experience certain limitations. While the conventional automated multiplanar imaging process permits a user to step through various view positions, the user is not afforded an easy manner to review previously considered view positions or exchange view positions. Instead, once a user moves onto the next view position, when it is desirable to review a previous view position, the user must repeat the steps necessary to re-create the prior view positions and re-enter the view mode. For example, the user must reposition the reference plane used as the basis to form the previous view position. Once the reference plane is re-created, the system recalculates the image planes associated with the reference plane.

A need remains for an improved method and system that affords an easy mechanism to return to previously viewed positions, and generally to move between pre-acquired view positions, without requiring reentry of the reference plane or other underlying information.

### BRIEF DESCRIPTION OF THE INVENTION

In accordance with an embodiment of the present invention, a diagnostic ultrasound system is provided for automatically displaying multiple planes from a 3-D ultrasound data set. The system comprises a user interface for designating a reference plane, wherein the user interface provides a safe view position option and a restore reference plane option. A processor module maps the reference plane into a 3D ultrasound data set and automatically calculates image planes based on the reference plane for a current view position and a prior view position. A display is provided to selectively display the image planes associated with the current and prior reference planes. Memory stores the prior reference plane in response to selection of the save reference plane option, while the display switches from display of the current reference plane to restore the prior reference plane in response to selection of the restore reference plane option. Optionally, the memory may store coordinates in connection with the current and prior reference planes.

Optionally, the user interface may include an auto sequence option that directs the display to sequentially display a series of image planes associated with the current view position. The display switches to a next image plane, in the series of image planes, each time the auto selection option is selected. Optionally, the display may simultaneously display multiple image planes that are aligned parallel to one another in connection with the current view position. Optionally, the user interface may include a marking option that permits a user to mark an image plane for storage or printing as a full-screen image. Optionally, the user interface may include a series of view buttons, each of which designates one of a series of view positions. The display displays the selected view position that corresponds to the selected one of the view buttons. The user interface may include shift and rotate commands that control linear and rotational movement of the reference plane horizontally/vertically and about at least one of the X, Y and Z axes, respectively. As a further option, the user interface may include a visualization mode command the controls the processor module to produce ultrasound images in one of a sectional planar image, volume rendered image, surface rendered image and a TUI image.

### BRIEF DESCRIPTION OF THE DRAWINGS

FIG. 1 illustrates a block diagram of a diagnostic ultrasound system formed in accordance with an embodiment of the present invention.

FIG. 2 illustrates a user interface having exemplary commands/options in accordance with an embodiment of the present invention.

FIG. 3 illustrates a command window presented on the display as part of the user interface for storing and restoring view positions in accordance with an embodiment of the present invention.

FIG. 4 illustrates a table storing view positions that define combinations of reference planes and auto image planes in accordance with an embodiment of the present invention.

FIG. 5 represents a graphical representation of different sets of image planes that may be stored and restored for display in accordance with an embodiment of the present invention.

FIG. 6 represents another graphical representation of different sets of image planes that may be stored and restored for display in accordance with an embodiment of the present invention.

FIG. 7 illustrates a processing sequence to store and restore view positions within an ultrasound 3-D data set in accordance with an embodiment of the present invention.

FIG. 8 illustrates a processing sequence to view image planes within a multiplanar data set in accordance with an embodiment of the present invention.

FIG. 9 illustrates a display format in which image planes may be presented in accordance with an embodiment of the present invention.

FIG. 10 illustrates a start screen that may be presented to the user on the touch screen at the beginning of a processing sequence.

FIG. 11 illustrates an exemplary pre-AMI mode display screen.

FIG. 12 illustrates an exemplary automatic multi-plane image (AMI) display screen.

### DETAILED DESCRIPTION OF THE INVENTION

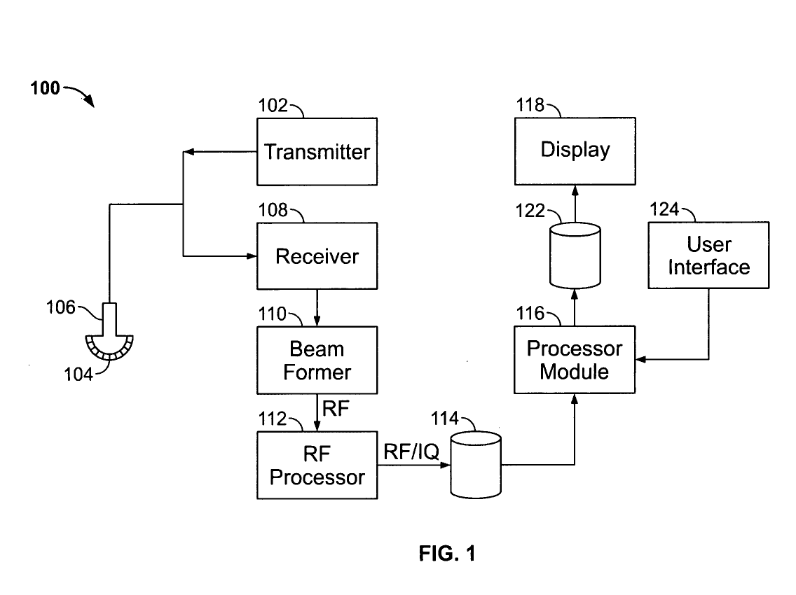

FIG. 1 illustrates a block diagram of an ultrasound system 100 formed in accordance with an embodiment of the present invention. The ultrasound system 100 includes a transmitter 102 which drives an array of elements 104 within a transducer 106 to emit pulsed ultrasonic signals into a body. A variety of geometries may be used. The ultrasonic signals are back-scattered from structures in the body, like blood cells or muscular tissue, to produce echoes which return to the elements 104. The echoes are received by a receiver 108. The received echoes are passed through a beamformer 110, which performs beamforming and outputs an RF signal. The RF signal then passes through an RF processor 112. Alternatively, the RF processor 112 may include a complex demodulator (not shown) that demodulates the RF signal to form IQ data pairs representative of the echo signals. The RF or IQ signal data may then be routed directly to memory 114 for storage.

The ultrasound system 100 also includes a processor module 116 to process the acquired ultrasound information (i.e., RF signal data or IQ data pairs) and prepare frames of ultrasound information for display on display 118. The processor module 116 is adapted to perform one or more processing operations according to a plurality of selectable ultrasound modalities on the acquired ultrasound information. Acquired ultrasound information may be processed in real-time during a scanning session as the echo signals are received. Additionally or alternatively, the ultrasound information may be stored temporarily in memory 114 during a scanning session and processed in less than real-time in a live or off-line operation. An image memory 122 is included for storing processed frames of acquired ultrasound information that are not scheduled to be displayed immediately. The image memory 122 may comprise any known data storage medium.

The processor module 116 is connected to a user interface 124 that controls operation of the processor module 116 as explained below in more detail. The display 118 includes one or more monitors that present patient information, including diagnostic ultrasound images to the user for diagnosis and analysis. The display 118 automatically displays multiple planes from the 3-D ultrasound data set stored in memory 114 or 122. One or both of memory 114 and memory 122 may store three-dimensional data sets of the ultrasound data, where such 3-D data sets are accessed to present 2-D and 3-D images. A 3-D ultrasound data set is mapped into the corresponding memory 114 or 122, as well as one or more reference planes. The position and orientation of the reference plane is controlled at the user interface 124.

The system 100 obtains volumetric data sets by various techniques (e.g., 3D scanning, real-time 3D imaging, volume scanning, 2D scanning with transducers having positioning sensors, freehand scanning using a Voxel correlation technique, 2D or matrix array transducers and the like). The transducer 106 is moved, such as along a linear or arcuate path, while scanning a region of interest (ROI). At each linear or arcuate position, the transducer 106 obtains scan planes that are stored in the memory 114.

FIG. 2 illustrates of the user interface 124 in more detail with exemplary commands/options afforded in accordance with an embodiment of the present invention. The user interface 124 includes a keyboard 126, a mouse 133, a touch screen 128, a series of soft keys 130 proximate the touch screen 128, a trackball 132, view position buttons 134, mode buttons 136 and keys 138. The soft keys 126 are assigned different functions on the touch screen 128 depending upon the examination made, stage of examination and the like. The trackball 132 and keys 138 are used to define a reference plane (e.g. designate an orientation and position of the reference plane, adjust the size and shape of the reference plane, shift and rotate the position of the reference plane relative to the reference coordinate system and the like). Once the reference plane is entered, the user selects an examination mode by entering one of the view position buttons 134. Each examination mode has one or more view positions, with respect to which one or more image planes is automatically calculated by the processor module 116. Optionally, the view position buttons 134 may be implemented as touch areas 129 on the touch screen 128. As a further option, the size, position and orientation of the reference plane may be controlled partially or entirely by touch areas provided on the touch screen 128 and/or by the soft keys 130.

The view position buttons 134 and examination modes may correspond to a four chamber view of a fetal heart, the right ventricular outflow, the left ventricular outflow, the ductal arch, the aortic arch, venous connections, the three vessel view and the like. The user interface 124 also includes a save reference plane command/option 140 and a restore reference plane command/option 142. The save reference plane command/option 140 directs the system 100 to save the coordinates associated with the reference plane. The restore reference plane option 142 directs the system 100 to switch the display from the display of a current reference plane to a prior reference plane.

The user interface 124 also include an auto sequence command/option 144 that directs the display 118 to sequentially display a series of image planes associated with the current view position. The display 118 switches to the next image plane in the series at image planes each time the auto selection option 144 is selected. Optionally, the display 118 may simultaneously co-display multiple image planes that are aligned parallel to one another within the 3-D ultrasound data set in connection with the current view position. Optionally, the user interface 124 may include a marking command/option 146 that permits a user to mark an image plane for storage or printing as a full-screen image. The user interface 124 may include shift and rotate command keys 138 and 139 that are used in combination with the trackball 132 to control linear and rotational movement of the reference plane horizontally/vertically and about at least one of the X, Y and Z axes, respectively. As a further option, the user interface 124 may include a visualization mode command 148 that controls the processor module 116 to produce ultrasound images in one of a sectional planar image, volume rendered image, surface rendered image and a TUI image.

The processor module 116 maps the reference plane into a 3-D ultrasound data set and automatically calculates image planes based on the reference plane for a current view position. The display 118 selectively displays the image planes associated with the current view position. The memory 114 or 122 stores the prior view position in response to selection of the save reference plane option 140, while the display 118 exchanges/switches from display of the current reference plane to the prior reference plane in response to selection of the restore reference plane option 142. Optionally, the memory 114, 122 may store, in connection with the current and prior reference plane, information other than coordinates of the associated reference plane and one or more image planes that collectively define the current view position and the prior view position.

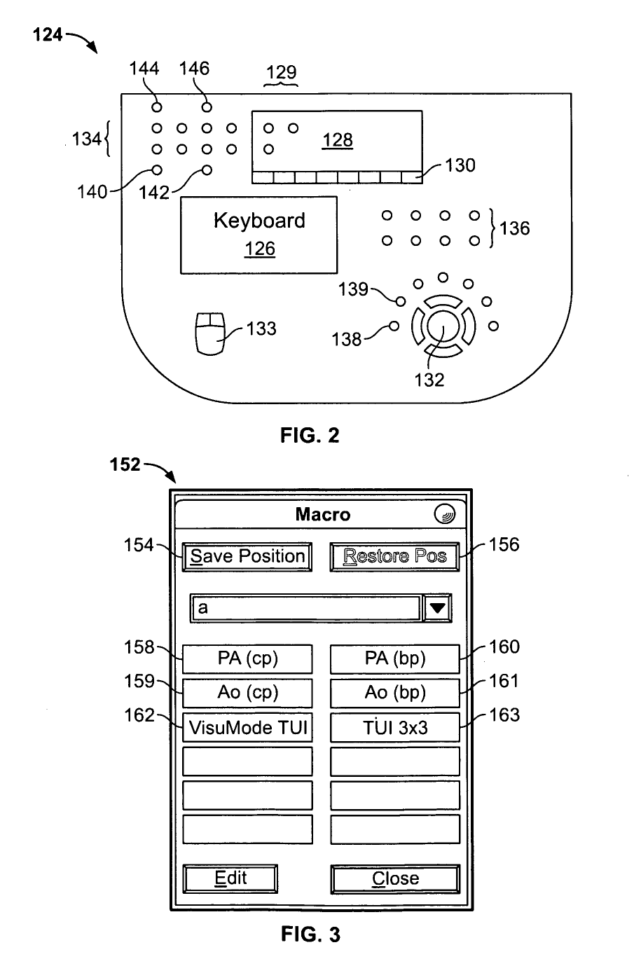

FIG. 3 illustrates a window 152 that may be presented on the display 118 and controlled by the mouse 133, the keyboard 126 and/or trackball 132 in accordance with an alternative embodiment of present invention. The window 152 includes virtual buttons such as a save reference plane option 154, and a restore reference plane option 156. The window 152 also includes reference plane adjustment options 158-161. The reference plane adjustment options 158-161 correspond to predefined combinations of shift and rotation operations to move the reference plane predetermined distances horizontally and vertically, as well as to rotate the reference plane by predetermined degrees. For example, option 158 may correspond to the forward shift by predetermined number of pixels or millimeters, while option 160 corresponds to a backward shift by a same predetermined number of pixels or millimeters. Options 159 and 161 may also correspond to forward and backward shifts, but in addition include rotations by predetermined number of degrees. The window 152 also includes a visualization mode option 162 and a TUI 3×3 option 163.

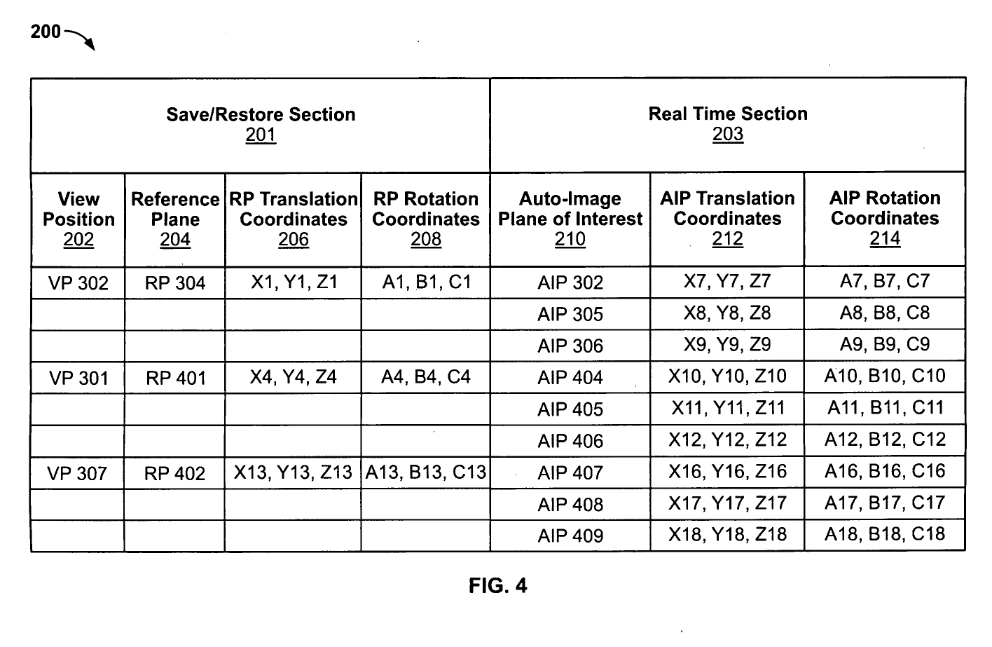

FIG. 4 illustrates a table 200, stored in memory 114 or 122. The table 200 is divided into a save/restore section 201 and a real-time section 203. The information in the save/restore section 201 may be stored and returned to while the information in the real-time section 203 is calculated while a set of image planes are calculated. The information in the real-time section 203 need not be saved. The save/restore section 201 stores predefined view positions 302, 3301 and 307. During operation, the user defines reference planes 304, 401 and 402, that are saved for subsequent reuse. Each reference plane 304, 401 and 402 is stored with a set of translation and rotation coordinates 206 and 208. Each view position 202 may be used with any of the reference planes 210.

Once a reference plane 204 and a view position 202 is selected, the system automatically calculates the image plane(s) 210 associated therewith and stored temporarily the corresponding translation and rotation coordinates 212 and 214. Each auto image plane 210 is defined in the table 200 by a series of translation and rotation coordinates 212 and 214, respectively. For example, view position 302 includes reference plane RP 304 which is defined by translation and rotation coordinates X1, Y1, Z1, A1, B1, C1. View position 302 also includes auto image planes (AIP) 303, 305 and which are defined by translation and rotation coordinates X7, Y7, Z7, A7, B7, C7, to X9, Y9, Z9, A9, B9, C9. Similarly, view position 301 includes reference plane 401 which is defined by translation and rotation coordinates X4, Y4, Z4, A4, B4, C4. View positions 301 also includes auto image planes (AIP) 404-406 which are defined by corresponding translation and rotation coordinates.

In the example of FIG. 4, the three-dimensional reference coordinate system is in Cartesian coordinates (e.g. XYZ). Thus, the translation coordinates 206, 212 represent translation distances along the X, Y and Z axes, while the rotation coordinates 208, 214 represent rotation distances about the X, Y and Z axes. The translation and rotation coordinates extend from/about an origin. Optionally, the 3D reference coordinate system may be in Polar coordinates.

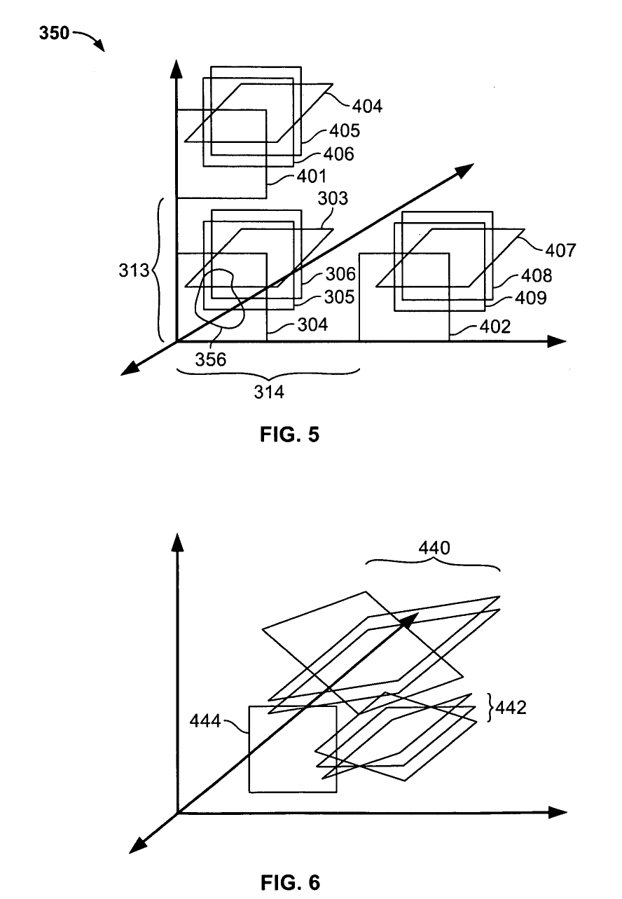

FIG. 5 represents a graphical representation of the reference planes and image planes of table 200 in FIG. 4. The image planes 303, 304, 305, 404-406, and 407-409 are automatically calculated from reference planes 304, 401 and 402. FIG. 5 illustrates a three-dimensional reference coordinate system 350, in which the reference plane 304 may be acquired as a single two-dimensional image (e.g. B-mode image or otherwise). Alternatively, the reference plane 304 may be acquired as part of a three-dimensional scan of a volume of interest. The reference plane 304 is adjusted and reoriented until the reference plane 304 contains a reference anatomy 356. Once the reference plane 304 is acquired, it is mapped into the 3-D reference coordinate system a 350. In the example of FIG. 5, the reference plane 304 is located at the origin. Optionally, reference plane 401 or 402 may be designated at distances 313 or 314 from the origin of the 3-D reference coordinate system 350 along the X, Y, and/or Z axes. After acquiring the reference plane 304 and after the user enters the desired view position 134, the processor module 116 automatically calculates additional image planes of interest, such as planes 303, 305 and 306. Alternatively, when reference plane 401 or 402 is defined, the processor module 116 automatically calculates image planes 404-406 or 407-409, respectively.

FIG. 6 represents another graphical representation of different sets of image planes 440 and 442 that may be automatically calculated from a common reference plane 444. The first set of image planes 440 is calculated when a first view position button 134 is selected, while the second set of image planes 442 is calculated when a different second view position button 134 is selected. Both sets of image planes 440 and 442 may be recalculated upon selection of the restore reference plane option 142.

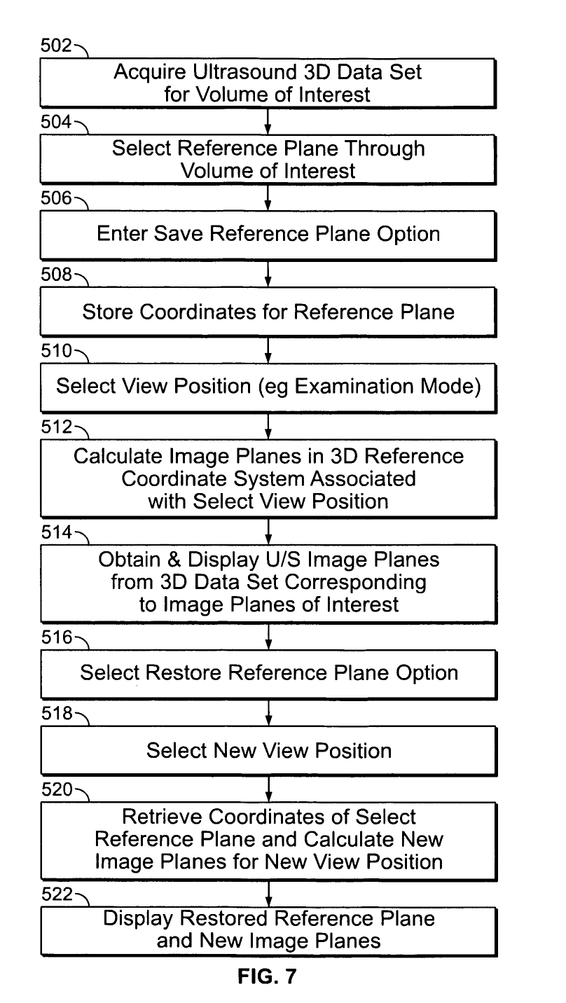

FIG. 7 illustrates a processing sequence to obtain ultrasound image planes from a pre-acquired 3-D data set in accordance with an embodiment of the present invention. Beginning at 502, a 3-D data set of ultrasound data is acquired for a volume of interest. At 504, the user selects a reference plane from the volume of interest. Once the user selects the reference plane, the reference plane may be mapped into a three-dimensional reference coordinate system. At 506, the user enters the “save reference plane option” and at 508, the system stores the coordinates of the reference plane in memory 200 (FIG. 4). At 510, the user selects the view position of interest which may also be defined as the examination mode. At 512, one or more image planes of interest are calculated within the three-dimensional reference coordinate system. At 514, ultrasound images, associated with the automatically calculated image planes, are obtained from the 3-D data set and presented as ultrasound images to a user in a desired format. At 516, the user selects a “restore reference plane option” and at 518 enters a new view position of interest. At 520, the system automatically calculates a new set of image planes associated with the restored reference plane and the newly selected view position. At 522, the restored reference plane is displayed and the newly calculated image planes are displayed.

The above operations may be repeated for the same reference plane, but for a different view position. Alternatively, the operations may be repeated for a different reference plane, but for the same view position. Alternatively, the operations may be repeated for a different reference plane, and for a different view position.

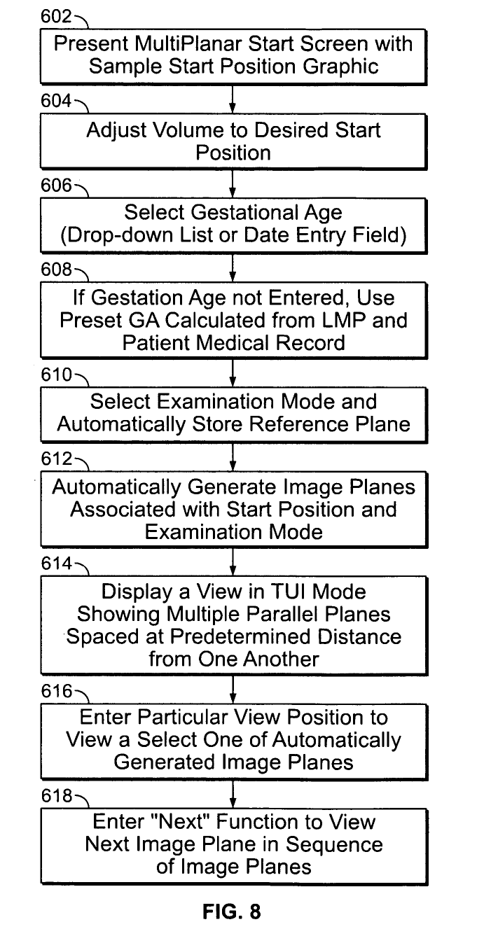

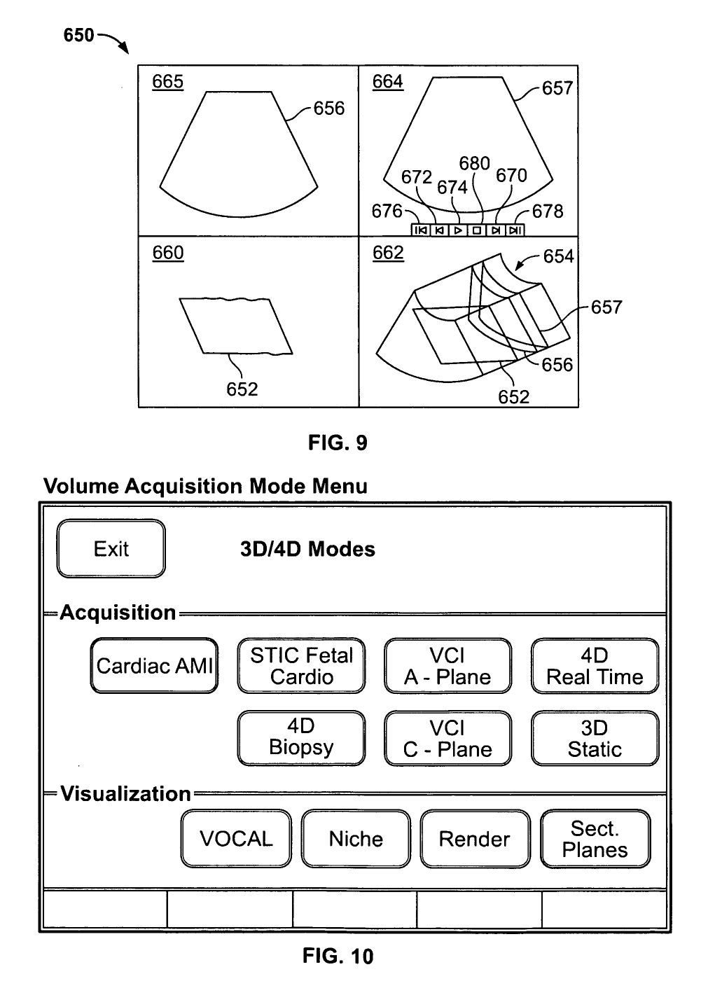

FIG. 8 illustrates a processing sequence of an alternative embodiment. Beginning at 602, a multiplanar start screen is present with a sample start position graphic. For examples, FIG. 9 illustrates an exemplary display 650 format having a sample start position graphic 652 overlaid upon the 3D data set 654. At 604, the user can adjust the volume, shape, size, orientation and position of the graphic 652 to the desired start position. The size and shape of the reference plane 652 may be changed in reference plane quadrant 660 by clicking and dragging on sides or corners of the reference plane 652. At 606, the user selects gestational age (e.g., from a drop down list or data entry field). At 608, when the gestational age is not entered, the user uses a preset GA (gestational age) calculated from the LMP and the patient medical record. At 610, the user selects examination mode by entering one of the view position buttons 134. At 610, the system automatically stores the reference plane that is being displayed when the examination mode is selected. Thus, the user need not manually enter a save reference plane option, but instead the save reference plane option is performed automatically. At 612, image planes, that are associated with the start position and examination mode, are automatically generated by the processor module 116. At 614, the user displays the view in TUI mode showing multiple parallel planes 656-657 spaced at a predetermined distance from one another. At 616, the user enters a particular view position to view a select one of automatically generated image planes. At 618, the user enters the “Next” function to view the next image plane in sequence of image planes.

As shown in FIG. 9, the display 650 has a reference plane quadrant 660 to control and manipulate the reference plane 652, a navigation quadrant 662 and image plane quadrants 664-665. The navigation quadrant 662 illustrates a model or actual 3D data set 654. Any number of image plane quadrants 664-665 may be presented, each of which shows one or more image planes 656-657 as 2D still, 2D cine, 2D color, 2D B-mode, 3D still, 3D cine, 3D color or 3D B-mode image planes.

Optionally, one or more of the quadrants 660-665 may include virtual page keys, such as a next plane key 670, a previous plane key 672, a plane cine loop key 674, a first plane key 676, a last plane key 678, and a stop cine loop key 680.

FIG. 10 illustrates a start screen that may be presented to the user on the touch screen 128 at the beginning of a processing sequence. The start screen is divided into an acquisition section, and a visualization section. Within the acquisition, the user is presented with different options such as “cardiac AMI”, STIC fetal cardio”, “VCI A-Plane”, “4D real time”, “4D biopsy”, “VCI C-plane” and “3D static”. Optionally, other visualization modes may be presented. In the screen of FIG. 10, the “cardiac AMI” mode is selected. Next, the user selected a visualization mode, such as vocal, niche, rendering, or select planes.

With reference to the flow charts of FIGS. 7 and 8, the start screen would be presented to the user at 502 or 602, respectively. In accordance with the process of FIG. 7, at 504, the user would select the select reference plane option from the start screen by entering the “Sect Planes”. In the example of FIG. 10, the select planes visualization mode has been selected indicating that the user desires to view a select set of image planes associated with the cardiac AMI examination mode.

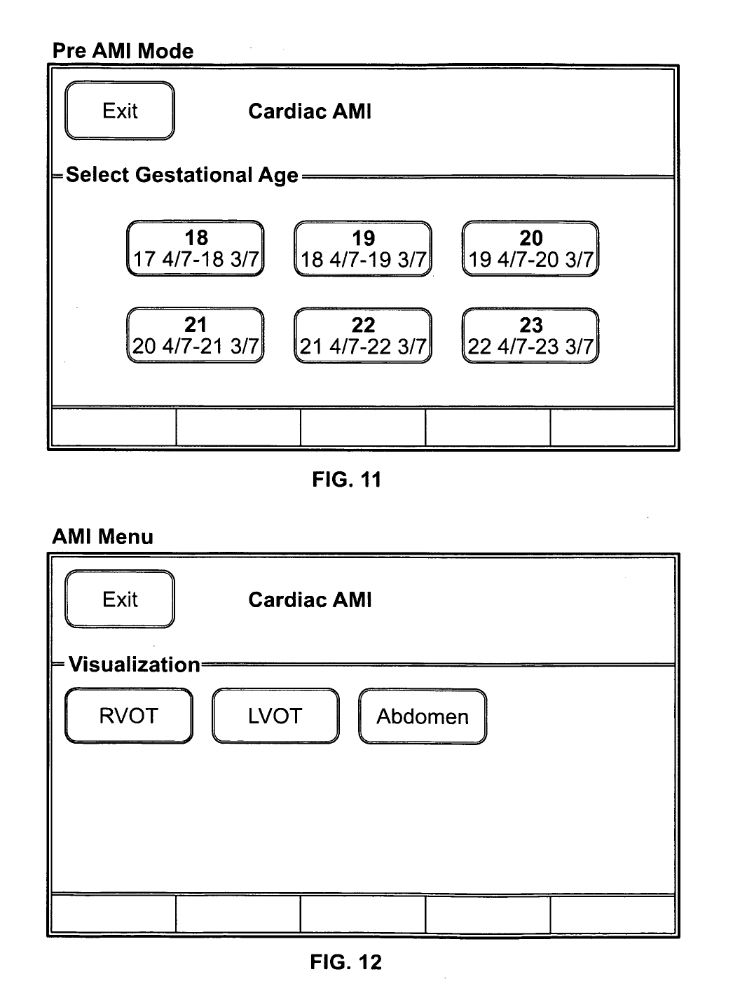

In the method of FIG. 8, once the user has selected the desired options from FIG. 10, flow passes to a new screen, such as presented in FIG. 11. FIG. 11 illustrates an exemplary pre-AMI mode display screen. In the pre-AMI mode display screen, the user is provided different gestational age options for a fetus, such as 18 weeks, 19 weeks, 20 weeks, 21 weeks and the like. The user enters the gestational age (in this example 18 weeks), which corresponds to 608 in FIG. 8, and flow moves to the screen shown in FIG. 12. Optionally, the options and screen of FIG. 10 may be omitted.

FIG. 12 illustrates an exemplary automatic multi-plane image (AMI) display screen. The AMI display screen is presented at 510 and 610 in the processes of FIGS. 7 and 8, respectively. The AMI display screen presents different view position options, such as right ventricular outflow (RVOT), left ventricular outflow (LVOT), and abdomen. In the example of FIG. 12, the user has selected the RVOT view position. Once a view position is selected, the processes of FIGS. 7 and 8 are completed in the manner described above.

While the invention has been described in terms of various specific embodiments, those skilled in the art will recognize that the invention can be practiced with modification within the spirit and scope of the claims.

What is claimed is:

1. A diagnostic ultrasound system for automatically displaying multiple planes from 3D ultrasound data set, the system comprising:
a user interface for designating a reference plane, wherein the user interface provides multiple predefined view positions.
a processor module mapping the reference plane into a 3D ultrasound data set, the processor module automatically calculates image planes based on the reference plane and relative a selected one of the predefined view positions;
a display selectively displaying the image planes associated with the reference plane and the selected predefined view position; and
memory storing coordinate information of the reference plane and relative coordinate information, with respect to the reference plane, of the predefined view positions.

2. The system of claim 1, wherein the coordinate information of the reference plane is stored automatically when selecting a first predefined view position.

3. The system of claim 1, wherein the coordinate information of the reference plane is stored according to a save reference plane option within a user interface.

4. The system of claim 1, wherein the reference plane is restored according to a restore reference plane option within a user interface.

5. The system of claim 1, wherein the user interface includes an auto-sequence option that directs the display to sequentially display a series of image planes associated with the current view position, the display switching to a next image plane in the series of image planes each time the auto-sequence option is selected.

6. The system of claim 1, wherein the display simultaneously displays multiple image planes aligned parallel to one another in connection with the current view position.

7. The system of claim 1, wherein the user interface includes a marking option that permits a user to mark an image plane for storage or printing as a full screen image.

8. The system of claim 1, wherein the user interface includes a shift command that controls linear movement of the reference plane horizontally and vertically.

9. The system of claim 1, wherein the user interface includes a rotate command that controls rotational movement of the reference plane about at least one of X, Y and Z coordinate axes.

10. The system of claim 1, wherein the user interface includes a visualization mode command controlling the processor module to produce ultrasound images in one of a sectional planar image, volume rendered image, surface rendered image, and a T.U.I. image.

11. A diagnostic ultrasound method for automatically displaying multiple planes from 3D ultrasound data set, the method comprising:
designating current and prior reference planes;
presenting, at a user interface, view position options, a save reference plane option and a restore reference plane option;
mapping the current and prior reference planes into a 3D ultrasound data set;
automatically calculating image planes based on the current and prior reference planes and view positions, the view positions being designated through selection of the view position options;
storing the prior reference plane in response to selection of the save reference plane option; and
selectively displaying the image planes associated with the current reference plane and a select view position, wherein, in response to selection of the restore reference plane option, the display switches from the current reference plane to restore the prior reference plane.

12. The method of claim 11, wherein the storing including storing coordinates in connection with each of the current and prior reference planes.

13. The method of claim 11, wherein the user interface includes an auto-sequence option that controls sequentially display of a series of image planes associated with the select view position, the displaying operation switching to a next image plane in the series of image planes each time the auto-sequence option is selected.

14. The method of claim 11, wherein the displaying operation simultaneously displays multiple image planes aligned parallel to one another in connection with the current reference plane.

15. The method of claim 11, further comprising providing, at the user interface, a marking option that permits a user to mark an image plane for storage or printing as a full screen image.

16. The method of claim 11, further comprising providing, at the user interface, a series of view buttons, each of the view buttons designating one of a series of view positions, the displaying including selecting the view positions that corresponds to the view button selected.

17. The method of claim 11, further comprising storing the current reference plane in response to selection of the save reference plane option.

18. The method of claim 11, further comprising providing, at the user interface, a shift command that controls linear movement of the reference plane horizontally and vertically.

19. The method of claim 11, further comprising providing, at the user interface, a rotate command that controls rotational movement of the reference plane about at least one of X, Y and Z coordinate axes.

20. The system of claim 11, further comprising providing, at the user interface, a visualization mode command controlling production of ultrasound images in one of a sectional planar image, volume rendered image, surface rendered image, and a T.U.I. image.
